(19) United States
(12) Patent Application Publication
(10) Pub. No.: US 2016/0143626 A1
(43) Pub. Date: May 26, 2016

(54) REMOTE INDICATION SUPPORT SYSTEM

(71) Applicant: FUJIFILM Corporation, Tokyo (JP)

(72) Inventors: Yasunori OHTA, Ashigarakami-gun (JP); Satoshi UEDA, Tokyo (JP); Ryosuke USAMI, Tokyo (JP)

(73) Assignee: FUJIFILM Corporation, Tokyo (JP)

(21) Appl. No.: 15/011,173

(22) Filed: Jan. 29, 2016

Related U.S. Application Data
(63) Continuation of application No. PCT/JP2014/070030, filed on Jul. 30, 2014.

(30) Foreign Application Priority Data
Jul. 31, 2013 (JP) 2013-159928

(51) Int. Cl. A61B 8/08; G08B 5/36; H04N 5/38; H04N 5/232; A61B 8/00; H04N 5/225

(52) U.S. Cl. CPC A61B 8/5207; A61B 8/4444; A61B 8/461; A61B 8/4263; H04N 5/2257; H04N 5/38; H04N 5/23293; G08B 5/36

(57) ABSTRACT

The remote indication support system includes an in-vehicle device which is mounted on an ambulance; and a remote indication device which is installed in a hospital. The in-vehicle device transmits a photographed image which has been photographed by a photographing unit, to the remote indication device through a communication network. The photographed image is displayed on a display, and an operating unit receives position designation of the inspection site of a patient within the photographed image. The remote indication device transmits an indication based on this position designation, to the in-vehicle device. The light pointer displaces the irradiation position of light based on the indication relating to the inspection site which has been received from the remote indication device.

### CROSS-REFERENCE TO RELATED APPLICATIONS

This application is a Continuation of PCT International Application No. PCT/JP2014/070030 filed on Jul. 30, 2014, which claims priority under 35 U.S.C §119(a) to Japanese Patent Application No. 2013-159928 filed Jul. 31, 2013. The above application is hereby expressly incorporated by reference, in its entirety, into the present application.

### BACKGROUND OF THE INVENTION

1. Field of the Invention

The present invention relates to a remote indication support system for indicating a method of operating a medical device, from a remote place.

2. Description Related to the Prior Art

In the medical field, a remote indication support system for indicating an operation method of a medical device from a remote place and supporting work of an operator of the medical device is known (refer to JP2006-115986A). The remote indication support system disclosed in JP2006-115986A includes a medical device which has an indication reception function of receiving an indication relating to an operation of the medical device; and a remote indication device which is connected to the medical device so as to be communicable through a communication network and transmits an indication to the medical device from a remote place. In JP2006-115986A, the medical device is inpatient's home, and a case in which a doctor in a hospital in a remote place which is away from the patient's home remotely indicates the operation method of the medical device with respect to the patient is exemplified.

The medical device is, for example, an ultrasound diagnostic apparatus. The ultrasound diagnostic apparatus has a probe which transceives an ultrasonic signal by being brought into contact with the body of a patient; a processer device generating an ultrasound image based on the ultrasonic signal which the probe has received; and a monitor displaying the ultrasound image generated in the processer device. The ultrasound diagnostic apparatus disclosed in JP2006-115986A has a function of transmitting an ultrasound image to a remote indication device and a doctor transmits an operation indication to the ultrasound diagnostic apparatus while looking at the sent ultrasound image. The ultrasound diagnostic apparatus is provided with a reception unit which receives an operation indication from the remote indication device; and an indication display unit which displays the received operation indication. A patient operates the ultrasound diagnostic apparatus while looking at the operation indication displayed on the indication display unit.

The content of the operation indication transmitted by the remote indication device is an operation method of a probe, and specifically, a message, such as “please apply the probe to the right abdominal region”, which indicates an inspection site to which the probe is applied. In addition, the remote indication device can receive an ultrasound image, and therefore, the doctor can guess the site, to which the probe is applied, from the ultrasound image. For this reason, in JP2006-115986A, an example of a message, such as “please move the probe 2 cm to the above”, which indicates the movement amount or the movement direction from the current position of the probe is disclosed in addition to an example of indicating the name of an inspection site as a message of indicating the inspection site. As a form of the indication display unit, a form of an indicator which is provided in the probe and indicates the movement direction is disclosed in addition to a form of using monitor on which the ultrasound image is displayed.

In recent years, prompt and easy ultrasonography using an ultrasound diagnostic apparatus is performed in order to promptly and easily perform initial diagnosis of trauma. This prompt and easy ultrasonography is called focused assessment with sonography for trauma (FAST). FAST is performed in order to perform an ultrasonic inspection on 6 inspection sites including the pericardium (an outer membrane which covers the heart), the right and left intercostals, the Morison's pouch (a region existing between the liver and the right kidney), the Douglas' pouch (a part of peritoneal cavity existing between the uterus and the rectum), and the periphery of the spleen and to check the presence and absence of a large amount of hemothorax caused by trauma to the abdominal region, intraperitoneal bleeding, cardiac tamponade (a state in which pulsation of the heart is inhibited due to a large amount of fluid stored between the heart and the pericardium), or the like. Moreover, FAST is performed as a primary inspection in order to determine a treatment policy, such as the necessity of an abdominal operation, after checking the presence and absence of fluid storage (bleeding) in the 6 sites.

FAST is used in the primary inspection, and therefore, it is preferable to perform FAST as early as possible after a patient is injured. For this reason, in some cases, FAST is performed by a rescue worker in an ambulance by operating an in-vehicle ultrasound diagnostic apparatus in a transport vehicle such as the ambulance while a patient is taken to the hospital from the scene of the accident. When a rescue worker performs FAST, in many cases, FAST is performed under an indication of a doctor by keeping in touch with a doctor in a hospital over a mobile phone in order to promptly and accurately execute FAST.

The remote indication support system disclosed in JP2006-115986A has merit that it is possible to visually check the name of an inspection site or the like through the indication display unit compared to a mobile phone. Therefore, it is considered that the remote indication support system is used inside an ambulance.

However, the average transportation time of a patient using an ambulance is about 30 minutes which is short, and therefore, promptness is required for work during the transportation. The remote indication support system disclosed in JP2006-115986A has a problem when being used in a scene, such as the inside of an ambulance, in which promptness is required. Moreover, the remote indication support system disclosed in JP2006-115986A displays a message on the indication display unit, and therefore, it is necessary for a rescue worker to receive an indication while alternately checking the indication display unit and the body of a patient. In addition, since the rescue worker is not as skilled as a doctor, there is also a problem in that it is difficult to intuitively grasp an inspection site from a message indicating the name of the inspection site or the movement direction or the movement amount of a probe. In this case, the rescue worker proceeds with the inspection while sequentially requiring confirmation from the doctor, and therefore, it is difficult to perform a prompt inspection.

The doctor who performs the remote indication needs to guess the current position of the probe from the ultrasound image. However, the ultrasound image is obtained by imaging the inside of the body of a patient and is not an image representing the outer shape of the body of the patient. Therefore, there is also a problem in that it takes time to grasp the current position of the probe from the ultrasound image. Particularly, in a case where the inspection site is a thin site of a part of an internal organ as in the case of FAST, such a problem becomes remarkable.

### SUMMARY OF THE INVENTION

An object of the present invention is to provide a remote indication support system which can promptly indicate an accurate position of an inspection site from a remote place when performing an inspection using a medical device in a transport vehicle transporting a patient.

In order to achieve the above-described object, a remote indication support system according to the present invention supports a remote indication which is related to an operation of a medical device and is performed from a remote place for an operator who operates the medical device. The remote indication support system includes an in-vehicle device in which the medical device is provided and which is provided within a transport vehicle for transporting a patient, and a remote indication device which is connected to the in-vehicle device through an communication network so as to be communicable with each other. The in-vehicle device has a photographing unit for photographing the patient; a photographed image transmission unit for transmitting a photographed image which has been photographed by the photographing unit to the remote indication device; an indication reception unit for receiving an indication relating to an inspection site, in which an inspection is performed by the medical device, from the remote indication device; and a light pointer for irradiating the patient with light so as to point to the inspection site, which is adapted to displace the position of emitting the light based on the indication which has been received by the indication reception unit. The remote indication device has a photographed image display unit for displaying the photographed image which has been received from the photographed image transmission unit; a position designation reception unit for receiving an input of a position designation operation for designating a position of the inspection site from a body of the patient within the photographed image; an indication generation unit for generating the indication based on the position designation operation received by the position designation reception unit; and an indication transmission unit for transmitting the indication generated in the indication generation unit to the in-vehicle device.

It is preferable that the remote indication support system further includes a coordinate transformation unit for transforming information of a coordinate within an image in the photographed image designated through the position designation operation, into information of an actual coordinate for controlling the position of emitting the light using the light pointer.

It is preferable that the light pointer has an irradiation unit for irradiating laser light, and a displacement mechanism for displacing the irradiation unit. In addition, it is preferable that the photographing unit is an optical camera which photographs the patient.

It is preferable that the medical device is an ultrasound diagnostic apparatus which has a probe to be brought into contact with the body of the patient, and generates and displays an ultrasound image based on a signal from the probe, and the inspection site is a site to be brought into contact with the probe. It is more preferable that the remote indication support system is used at the time of performing FAST, as prompt and easy ultrasonography, using the ultrasound diagnostic apparatus.

It is preferable that the remote indication device has a current position reception unit for receiving a current position of the position of emitting the light using the light pointer, from the in-vehicle device, and the current position is superimposed on the photographed image and displayed in the photographed image display unit.

It is preferable that the in-vehicle device has a damping device which is provided to a bed fixing base for fixing a bed, on which the patient is laid down, and removes a vibration transmitted to the bed from the transport vehicle. It is more preferable that at least one of the photographing unit and the light pointer is fixed to the bed fixing base.

According to the present invention, it is possible to control the position of emitting light using the light pointer within the transport vehicle, through position designation within a photographed image in a remote place. Therefore, it is possible to accurately provide an indication of the inspection position to an operator of the medical device within the transport vehicle. For this reason, it is possible to provide the remote indication support system which can promptly indicate the accurate position of the inspection site from the remote place when performing an inspection using the medical device within the transport vehicle transporting a patient.

### BRIEF DESCRIPTION OF DRAWINGS

For more complete understanding of the present invention, and the advantage thereof, reference is now made to the subsequent descriptions taken in conjunction with the accompanying drawings, in which:

FIG. 1 is a schematic view of a first embodiment of a remote indication support system according to the present invention;

FIG. 2 is a schematic view of an ultrasound diagnostic apparatus used in the present invention;

FIG. 3 is an explanatory view relating to inspection positions of FAST;

FIG. 4 is a detailed explanatory view of the first embodiment;

FIG. 5 is a functional view of the first embodiment;

FIG. 6 is an explanatory view relating to coordinate transformation in the first embodiment;

FIG. 7 is a flowchart view of the first embodiment;

FIG. 8 is an explanatory view of a second embodiment of a remote indication support system according to the present invention;

FIG. 9 is an explanatory view of a third embodiment of a remote indication support system according to the present invention; and

FIG. 10 is an explanatory view relating to coordinate transformation in the third embodiment.

### DESCRIPTION OF THE PREFERRED EMBODIMENTS

### First Embodiment

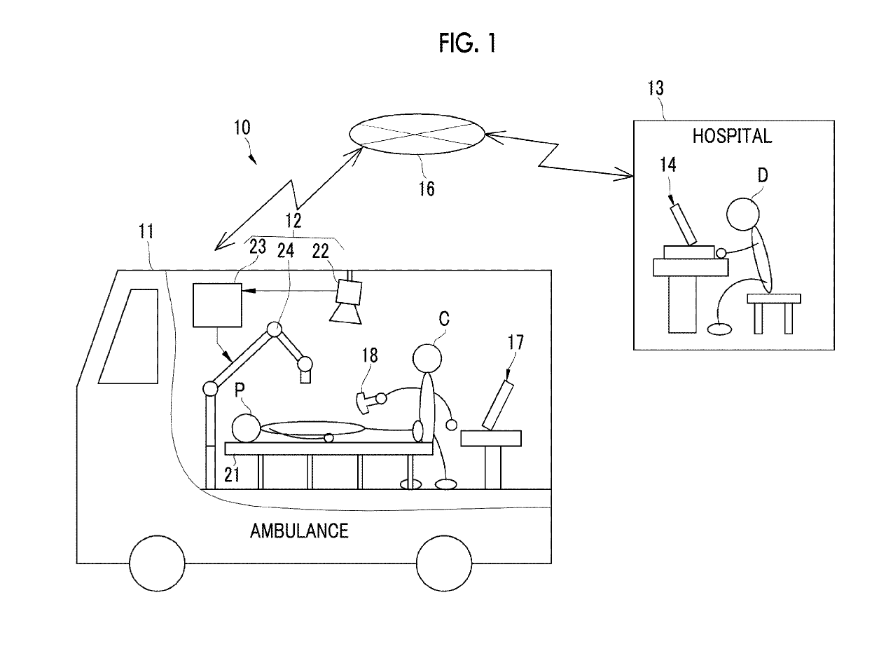

The detail of a first embodiment of a remote indication support system of the present invention will be described below. As shown in FIG. 1, a remote indication support system 10 includes an in-vehicle device 12 which is mounted on an ambulance 11; and a remote indication device 14 which is installed in a medical institution such as a hospital 13. The in-vehicle device 12 and the remote indication device 14 are connected to each other so as to be communicable through a communication network 16 such as a mobile communication network or a wireless wide area network (wireless WAN). An ultrasound diagnostic apparatus 17 which is used in a primary inspection of a patient P who has received trauma is mounted on the ambulance 11 as well. On the inside of the ambulance 11 while transporting the patient P to the hospital 13 which is a transportation destination, a rescue worker C performs FAST, which is prompt and easy ultrasonography as a primary inspection, on the patient P by operating the ultrasound diagnostic apparatus 17. The remote indication support system 10 is used for the rescue worker C to receive an operation indication with respect to an operation method of the ultrasound diagnostic apparatus 17 from a doctor D who is in the remote hospital 13.

The ultrasound diagnostic apparatus 17 has a probe 18 which transceives an ultrasonic signal for generating an ultrasound image, by being applied to an inspection site of the body of a patient P. The rescue worker C receives an indication relating to the inspection site to which the probe 18 is applied, from the doctor D through the remote indication support system 10. The remote indication device 14 is operated by the doctor D and transmits the indication relating to the inspection site, to which the probe 18 is applied, to the in-vehicle device 12.

The in-vehicle device 12 has a photographing unit 22 which outputs a photographed image by photographing the body of the patient P who is laid down on a bed such as a stretcher 21; a control device 23 which transmits the photographed image, output by the photographing unit 22, to the remote indication device 14 via a communication network 16, and receives an operation indication from the remote indication device 14; and a light pointer 24 which points to the inspection site, to which the probe 18 is applied, by irradiating the body of the patient P with light. The light pointer 24 is controlled by the control device 23 and can displace the position of the body to be irradiated with light, based on the operation indication received by the control device 23. The photographing unit 22 is an optical camera for photographing the patient P under visible light, and records a photographed image as digital data. The photographing unit 22 is fixed to the ceiling of the inside of the ambulance 11 so as to photograph an overlooking image of the body of the patient P. The photographed image is, for example, a moving image and can inform the doctor D of the condition of the patient P in the ambulance 11 in real time, using the photographing unit 22.

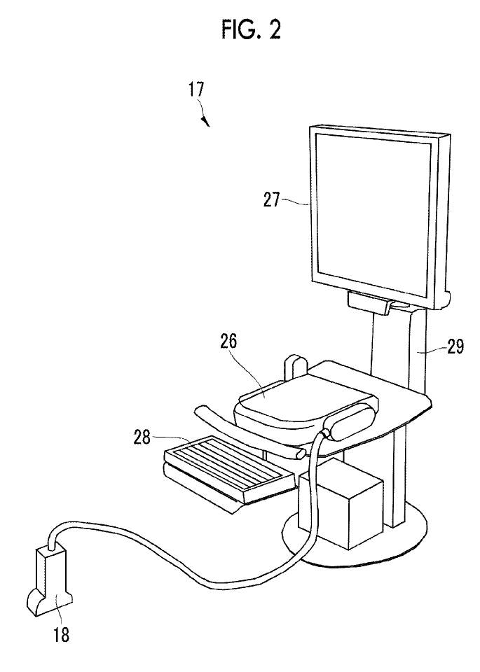

As shown in FIG. 2, the ultrasound diagnostic apparatus 17 has the probe 18; a processer device 26 generating an ultrasound image, which is a tomographic image in the patient P, based on an ultrasonic signal received by the probe 18; a monitor 27 which displays the ultrasound image generated in the processer device 26; and an operating unit 28. The processer device 26, the monitor 27, and the operating unit 28 are mounted on the ambulance 11 in a state of, for example, being accommodated in a rack 29. The probe 18 is connected to the processer device 26 using a flexible communication cable through which communication of a control signal from the processer device 26 or an ultrasonic signal toward the processer device 26 is performed.

The processer device 26 is connected to the in-vehicle device 12 so as to be communicable through a wire or wirelessly. An ultrasound image generated by the processer device 26 is output to the monitor 27 and is transmitted to the in-vehicle device 12.

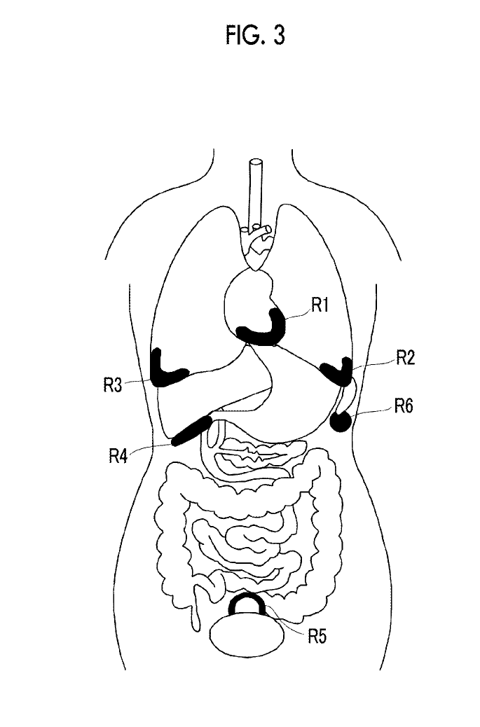

As shown in a schema (a schematic anatomical view of a human body) of FIG. 3, inspection sites when performing FAST are 6 sites including the pericardium (an outer membrane which covers the heart) R1, the right and left intercostals R2 and R3, the Morison's pouch (a region existing between the liver and the right kidney) R4, the Douglas' pouch (a part of peritoneal cavity existing between the uterus and the rectum) R5, and the periphery of the spleen R6. In FAST, an ultrasonic inspection is executed by sequentially applying the probe 18 to these 6 inspection sites, and the presence and absence of a large amount of hemothorax caused by trauma to the abdominal region, intraperitoneal bleeding, cardiac tamponade (a state in which pulsation of the heart is inhibited due to a large amount of fluid stored between the heart and the pericardium) is checked. Moreover, it is possible to evaluate the necessity of an abdominal operation using observations of fluid storage (bleeding) in the 6 sites. In order to appropriately perform FAST, it is necessary to accurately apply the probe 18 to an inspection site for FAST.

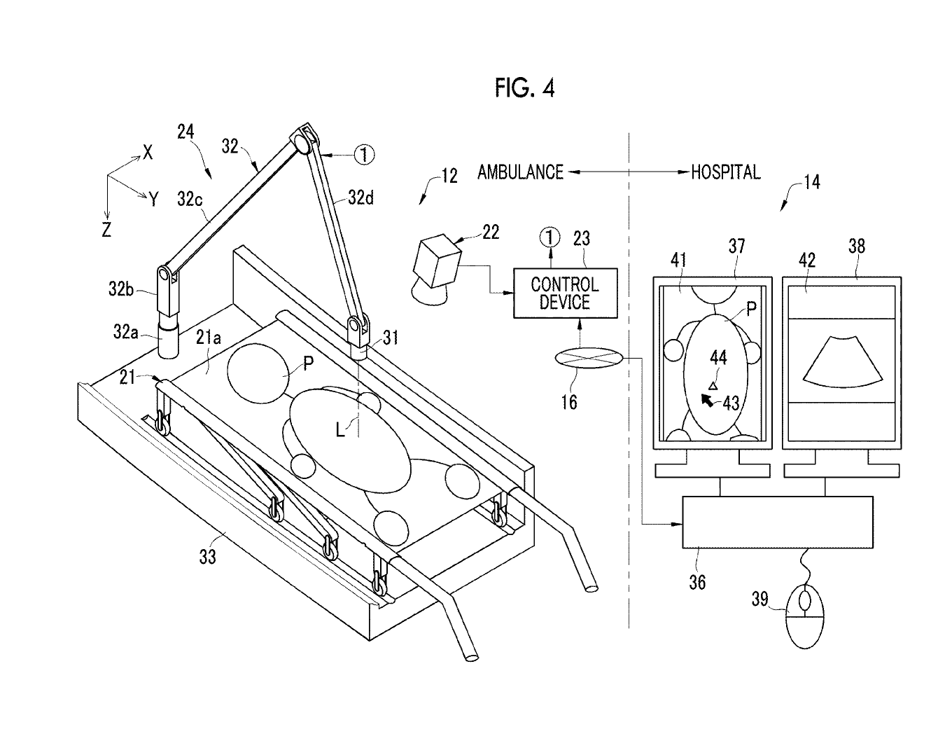

In FIG. 4, the light pointer 24 is a so-called laser pointer which informs the rescue worker C of the position to which the probe 18 is applied, by pointing to an inspection site by irradiating the body of the patient P with laser light L. Regarding the position of emitting laser light L using the light pointer 24, a remote operation performed by the doctor ID can be carried out using the remote indication device 14. It is possible to sequentially indicate the inspection site for the rescue worker C by moving the irradiation position of laser light to the inspection site through the remote operation of the doctor D.

The light pointer 24 has an irradiation unit 31 which emits laser light; and a displacement mechanism 32 which displaces the direction of the irradiation unit 31 in three axial directions of an X axis, a Y axis, and a Z axis. The irradiation unit 31 has a laser light source which is constituted of a semiconductor element. The displacement mechanism 32 is constituted of a pedestal 32a which is fixed to a bed fixing base 33 to which the stretcher 21 is fixed; a supporting post 32b which is provided in the pedestal 32a and is rotatable around the Z axis; and two arms 32c and 32d. End portions of the arm 32c and the arm 32d are rotatably attached to the periphery of an axis orthogonal to the Z axis direction, and constitute an arm unit which is bendable in a V shape. One end of the arm 32c is attached to the supporting post 32b and the arm 32c is also rotatable with respect to the supporting post 32b in the axial direction orthogonal to the Z axis. The irradiation unit 31 is attached to a distal end of the arm 32d and is also rotatable in the axial direction orthogonal to the Z axis.

The displacement mechanism 32 moves the irradiation unit 31 to an arbitrary position within an X-Y plane parallel to the top plane of a mat portion 21a (a portion on which the patient P is laid down) of the stretcher 21, through a rotation of the supporting post 32b, the arms 32c and 32d, and the irradiation unit 31. The supporting post 32b, the arms 32c and 32d, and the irradiation unit 31 is electrically rotated by a driving mechanism (not shown) constituted of a motor, a wire, or the like. Lighting on and off of the irradiation unit 31 or an operation of the displacement mechanism 32 is controlled by the control device 23. The position of emitting laser light L using the irradiation unit 31 can be moved to an arbitrary position of the body of the patient P lying on the mat portion 21a using the displacement mechanism 32.

The remote indication device 14 is a device in which an operating system or application software, such as software for a remote indication, is installed based on a personal computer or a workstation which is constituted of hardware such as a central processing unit (CPU: central (arithmetic) processing unit), a memory, and a communication circuit. The remote indication device 14 is constituted of a main body portion 36, two displays 37 and 38, and an operating unit 39. The main body portion 36 is a control unit which controls the remote indication device 14. One display 37 functions as a photographed image display unit which displays a photographed image 41 output from the photographing unit 22. Another one display 38 displays an ultrasound image 42 output from the ultrasound diagnostic apparatus 17. The operating unit 39 is constituted of a mouse, a keyboard, or the like, and inputs an operation signal to the main body portion 36.

A pointer 43 is displayed within the photographed image 41 displayed on the display 37. The position of the pointer 43 is operated by the operating unit 39. A position designation operation of designating the position within the photographed image 42 is performed through an operation of the pointer 43. Specifically, the position designation operation is an operation of determining the position of the pointer 43 through a click operation of a mouse, and an operation of depressing a return key of a keyboard, by moving the pointer 43 to an arbitrary position of the body of the patient P which is projected on the photographed image 41. A mark 44 having, for example, a triangular shape is displayed at the position which is determined by the position designation operation.

The main body portion 36 receives an input of a position designation operation; generates a movement indication for moving the position of emitting laser light using the irradiation unit 31 of the light pointer 24, based on the designated position; and transmits the generated movement indication to the in-vehicle device 12 via the communication network 16. Here, the position designation operation is a position designation operation for designating the position of an inspection site from the body of the patient P within the photographed image 41, and the movement indication is an indication relating to an inspection site in which an inspection is performed by the ultrasound diagnostic apparatus 17.

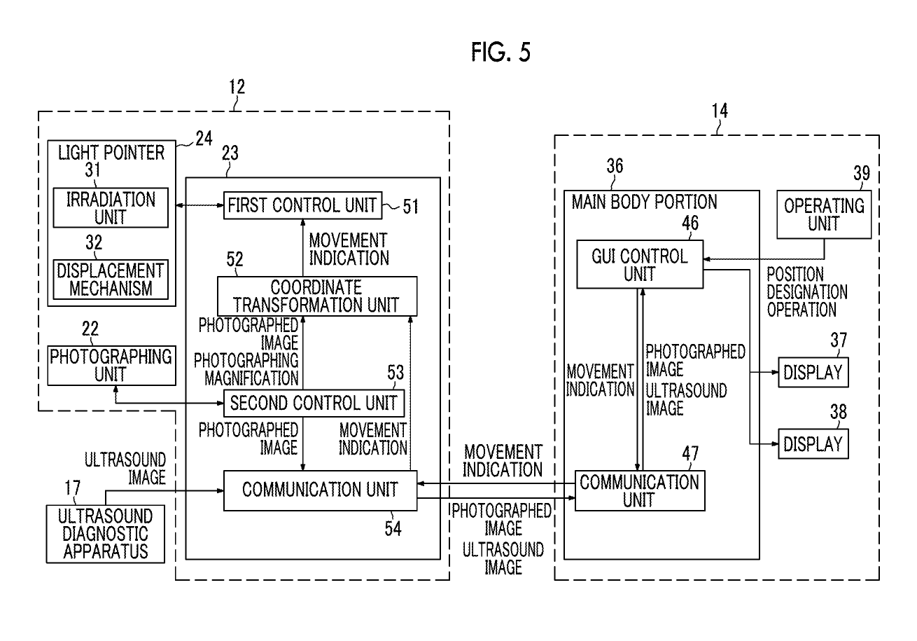

In FIG. 5, the main body portion 36 of the remote indication device 14 has a graphical user interface (GUI) control unit 46 and a communication unit 47. The GUI control unit 46 and the communication unit 47 are realized by cooperation with hardware such as a CPU, a memory, and a communication circuit, an operating system, and software for a remote indication. The communication unit 47 receives the photographed image 41 and the ultrasound image 42 which are sent from the in-vehicle device 12 and input the received images to the GUI control unit 46. In addition, the communication unit 47 transmits a movement indication input from the GUI control unit 46 to the in-vehicle device 12 through the communication network 16. The communication unit 47 functions as an indication transmission unit.

The GUI control unit 46 displays the pointer 43 or an operation screen including various operation commands on the displays 37 and 38, and receives an input of an operation of a user which contains a position designation operation from the operation screen and the operating unit 39. In addition, the GUI control unit 46 also functions as a display control unit which displays the photographed image 41 or the ultrasound image 42 which has been received by the communication unit 47, on each of the displays 37 and 38.

When an input of a position designation operation is received, the GUI control unit 46 specifies a coordinate (hereinafter, referred to as a coordinate within an image) within a photographed image 41 corresponding to the designated position, and generates a movement indication in which the movement destination of the irradiation unit 31 is designated by the specified coordinate within an image. The GUI control unit 46 inputs the generated movement indication to the communication unit 47. In this manner, the GUI control unit 46 functions as a position designation reception unit and an indication generation unit.

The control device 23 has a first control unit 51 which controls the light pointer 24; a coordinate transformation unit 52; a second control unit 53 which controls the photographing unit 22; and a communication unit 54. The first control unit 51 controls lighting on and off of the irradiation unit 31 of the light pointer 24. In addition, the first control unit 51 controls the irradiation position of the irradiation unit 31 by operating the displacement mechanism 32 based on a movement indication received from the remote indication device 14.

Specifically, the first control unit 51 grasps the current position of the irradiation unit 31 by detecting the amount of rotation of a motor which drives the supporting post 32b, the arms 32c and 32d, and the irradiation unit 31. The current position is represented by an actual coordinate within an X-Y plane. The actual coordinate is represented by the amount of displacement of an X direction and a Y direction from a reference position by having, for example, the central position in the X-Y plane of the mat portion 21a as the reference position. The first control unit 51 controls the irradiation position of the irradiation unit 31 based on the movement indication which has been input from the coordinate transformation unit 52. In the movement indication at a point in time of being transmitted from the remote indication device 14, the movement destination is designated by a coordinate within an image. However, as will be described below, the coordinate within an image, which is included in the movement indication, is transformed into an actual coordinate by the coordinate transformation unit 52 and is input to the first control unit 51.

The second control unit 53 controls the start and the completion of photographing of the photographing unit 22 and receives a photographed image from the photographing unit 22. The second control unit 53 transmits the photographed image 41 to the remote indication device 14 through the communication unit 54. The communication unit 54 transmits the photographed image 41 and an ultrasound image, which has been received from the ultrasound diagnostic apparatus 17, to the remote indication device 14. In addition, the communication unit 54 receives a movement indication from the remote indication device 14. In this manner, the communication unit 54 functions as an indication reception unit and a photographed image transmission unit. In addition, the second control unit 53 inputs the photographed image 41 and a photographing magnification (a ratio of the size of an actual photographic subject to the size of a photographic subject within the photographed image 41) of the photographing unit 22, into the coordinate transformation unit 52.

The coordinate transformation unit 52 transforms designation of the movement destination included in the movement indication transmitted from the remote indication device 14, from the coordinate within an image into the actual coordinate based on the photographed image and the photographing magnification. Then, the movement indication after the transformation in which the movement destination is designated by the actual coordinate is input to the first control unit 51. The central position of the angle of view of the photographing unit 22 is set so as to coincide with the central position of the mat portion 21a similarly to the reference position of the irradiation unit 31.

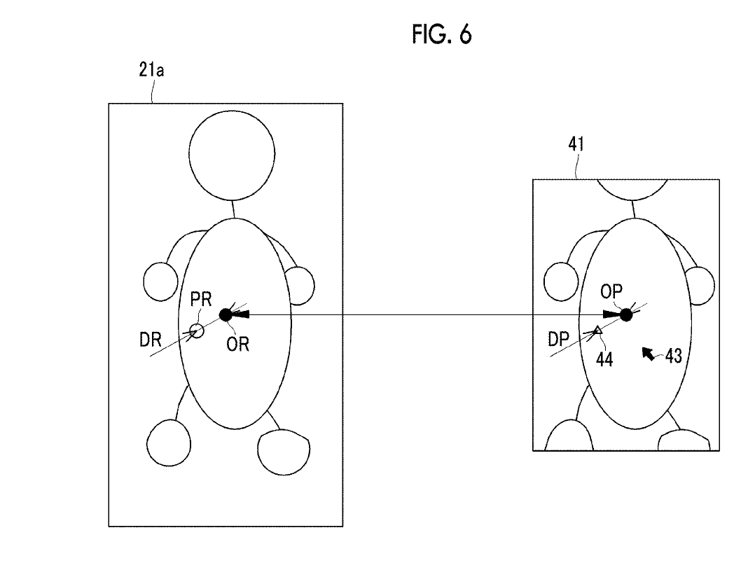

For this reason, as shown in FIG. 6, a central position OP of the photographed image 41 which is photographed by the photographing unit 22 and a central position OR of the mat portion 21a coincide with each other. Moreover, if the photographing magnification is known, it is possible to transform the distance within the image within the photographed image 41 into the actual distance on the mat portion 21a, and therefore, it is possible to transform the designation from the coordinate within an image into the actual coordinate. For example, in a case where the current position of the irradiation unit 31 is the central position OR which is a reference position, the central position OP of the photographed image 41 and the irradiation position of the irradiation unit 31 correspond to each other. In the remote indication device 14, in a case where the position shown by the mark 44 of the photographed image 41 is designated as a movement destination of the irradiation unit 31 through a position designation operation, the coordinate within an image of the position of the mark 44 is specified. When the coordinate transformation unit 52 receives a movement indication designated by the coordinate within an image, the coordinate transformation unit calculates the movement direction and the movement distance DP with respect to the central position OP of the photographed image 41 based on the coordinate within an image corresponding to the mark 44. The movement distance DP is transformed into the movement distance DR on the actual coordinate of the mat portion 21a when the photographing magnification is multiplied by the movement distance DP. The actual coordinate of the movement destination PR with respect to the central position OR of the mat portion 21a is calculated based on the movement distance DR and the movement direction.

As the method of transforming the designation from the coordinate within an image into the actual coordinate of this example, an example is assumed in which any of four corners of the screen of the photographed image 41 such as an upper left corner of the screen is set as a reference position, in relation to the coordinate within an image. In this case, the reference position of the actual coordinate in the mat portion 21a and the reference position of the coordinate within an image do not coincide with each other. Therefore, coordinate transformation is performed by once obtaining the movement distance and the movement direction based on the coordinate within an image and the actual coordinate. However, various modes can be considered as the method of taking the reference position of the actual coordinate or the coordinate within an image, and therefore, it is possible to employ an appropriate coordinate transformation method in accordance with the method of taking the reference position. For example, in a case where the coordinate within an image is represented by the amount of displacement of the X direction and the Y direction in which the central position OP within the photographed image 41 is set as a reference position similarly to the actual coordinate, the actual coordinate is obtained by simply multiplying the photographing magnification by the coordinate within an image.

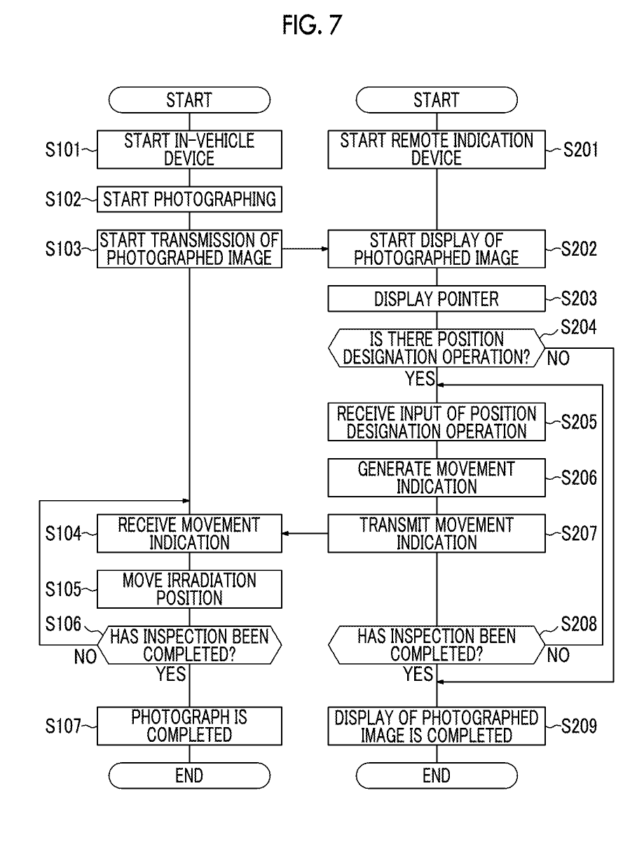

Hereinafter, the action of the above-described configuration will be described based on a flowchart shown in FIG. 7. In the ambulance 11, after the patient P is laid down by the rescue worker C on the mat portion 21a, the in-vehicle device 12 is started by the rescue worker C (in-vehicle device start step S101). Accordingly, the first control unit 51 within the control device 23 starts control of the light pointer 24. In addition, the second control unit 53 starts control of the photographing unit 22. In contrast, in the hospital 13, the remote indication device 14 is started by the doctor U (remote indication device start step S201).

The second control unit 53 starts photographing of the body of the patient P using the photographing unit 22 (photographing start step S102). Accordingly, the photographing unit 22 starts acquisition of a photographed image 41. The photographed image 41 is sent to the communication unit 54 from the photographing unit 22 via the second control unit 53. The communication unit 54 starts transmission of the photographed image 41 to the remote indication device 14 through the communication network 16 (photographed image transmission start step S103). Thereafter, the communication unit 54 continuously transmits the photographed image 41 to the remote indication device 14 until the photographing of the patient P using the photographing unit 22 is completed.

The communication unit 47 of the remote indication device 14 starts reception of the transmitted photographed image 41. The received photographed image 41 is transmitted to the GUI control unit 46 from the communication unit 47. The GUI control unit 46 starts display of the photographed image 41 on the display 37 (photographed image display start step S202). Thereafter, the GUI control unit 46 continuously displays the photographed image 41 on the display 37 until the reception of the photographed image 41 is completed. In addition, the GUI control unit 46 displays the pointer 43 within the photographed image 41 displayed on the display 37 (pointer display step S203).

In a case where the doctor D determines that it is necessary to indicate an inspection site for the rescue worker C, a position designation operation is performed by the doctor D who operates the remote indication device 14 (YES in position designation operation determination step S204). The GUI control unit 46 receives an input of the position designation operation (position designation operation input reception step S205).

The GUI control unit 46, which has received the input of the position designation operation, specifies a coordinate within an image corresponding to the designated position and generates a movement indication in which the movement destination of the irradiation unit 31 is designated by the specified coordinate within an image (movement indication generation step S206). The GUI control unit 46 displays the triangular mark 44 on the specified coordinate within an image.

The generated movement indication is transmitted to the communication unit 47 from the GUI control unit 46. The communication unit 47 transmits the received movement indication to the in-vehicle device 12 through the communication network 16 (movement indication transmission step S207). The communication unit 54 of the in-vehicle device 12 starts reception of the transmitted movement indication (movement indication reception step S104). The received movement indication is transmitted to the coordinate transformation unit 52 from the communication unit 54.

The coordinate transformation unit 52 receives an input of the photographed image 41 and the photographing magnification of the photographing unit 22 from the second control unit 53 in accordance with the reception of the movement indication. The coordinate transformation unit 52 transforms designation of a movement destination included in the movement indication from the coordinate within an image into an actual coordinate, based on the photographed image 41 and the photographing magnification. Then, the movement indication after the transformation in which the movement destination is designated by the actual coordinate is input to the first control unit 51. The first control unit 51 moves the irradiation unit 31 by operating the displacement mechanism 32 based on the movement indication in the actual coordinate (irradiation position movement step S105). Thereafter, the first control unit 51 makes the irradiation unit 31 emit laser light.

Accordingly, the rescue worker C can photograph an ultrasound image by applying the probe 18 to a site of the patient P with which laser light is irradiated by the irradiation unit 31. The photographed ultrasound image is displayed on the monitor 27 and is transmitted to the communication unit 54. The communication unit 54 transmits the ultrasound image to the remote indication device 14 through the communication network 16. This ultrasound image is sent to the GUI control unit from the communication unit 47 and is displayed on the display 38. In this manner, an ultrasonic inspection is performed on a site based on the indication of the doctor.

Furthermore, when performing the ultrasonic inspection (No in inspection completion determination steps S208 and S106), the position designation operation input reception step S205 to movement indication transmission step S207, movement indication reception step S104 to irradiation position movement step S105, and the ultrasonic inspection are performed again.

In contrast, in a case where it is unnecessary to further perform the ultrasonic inspection (NO in inspection completion determination steps S208 and S106), the ultrasonic inspection is completed. The second control unit 53 completes the photographing of the body of the patient P using the photographing unit 22 (photographing completion step S107). In addition, the GUI control unit 46 completes the display of the photographed image 41 on the display 37 (photographed image display completion step S209).

In addition, in position designation operation determination step S204, in a case where the doctor D determines that it is unnecessary to indicate the inspection site for the rescue worker C (No in S204), photographing completion step S107 and the photographed image display completion step S209 are performed similarly to the case of being YES in inspection completion determination steps S208 and S106.

The present invention is made such that the irradiation position of laser light using the light pointer 24 within the ambulance 11 can be controlled by designating the position within the photographed image 41 performed by the doctor D from the hospital 13 which is a remote place. Therefore, it is possible to accurately provide an indication of the inspection position to the rescue worker C within ambulance 11. For this reason, the doctor D can promptly indicate an accurate position of an inspection site from a remote place. In the present invention, it is possible to indicate an accurate inspection position using laser light, and therefore, the present invention is particularly effectively used when performing inspection such as the ultrasonic inspection within a comparatively narrow range of the inspection position.

In addition, the indication of the doctor D is performed by emitting laser light, and therefore, the rescue worker C can intuitively grasp the indication of the doctor D. For this reason, the present invention is particularly effectively used when a prompt inspection is required. In the case of FAST which requires an ultrasonic inspection of a plurality of sites (6 sites) in a short period of time (about 30 minutes), the present invention is particularly effectively used.

In the first embodiment, the coordinate transformation unit 52, which transforms the designation of the movement destination included in the movement indication from the coordinate within an image into the actual coordinate, is provided in the in-vehicle device 12. However, the coordinate transformation unit 52 may be provided in the remote indication device 14.

In the first embodiment, the triangular mark 44 is displayed at a position which is designated through the position designation operation on the photographed image 41 within the display 37. Accordingly, the doctor D can confirm the position to be designated by the position designation operation, and therefore, it is easy to perform the position designation operation. In addition to this, or instead of this, the current position of the irradiation position of laser light may be received from the in-vehicle device 12 to be displayed on the photographed image 41 in an overlapping manner. The doctor D can confirm the current position by displaying the current position, and therefore, the present invention is easily used.

In the first embodiment, an optical camera using visible light has been used for the photographing unit 22. However, an infrared camera using infrared light may be used instead of the optical camera.

In addition, an irradiation unit having a laser light source has been used in the first embodiment as the irradiation unit 31. However, instead of this, an irradiation unit having a light source for emitting light with directivity or convergence properties may be used. In addition, an arm-like displacement mechanism has been used for the displacement mechanism 32. However, any mode in which the irradiation unit 31 is moved to a designated movement destination may be used. For example, a mode, in which a frame is provided with an actuator on the top of a bed fixing base and the irradiation unit 31 is moved by the actuator, may be used.

### Second Embodiment

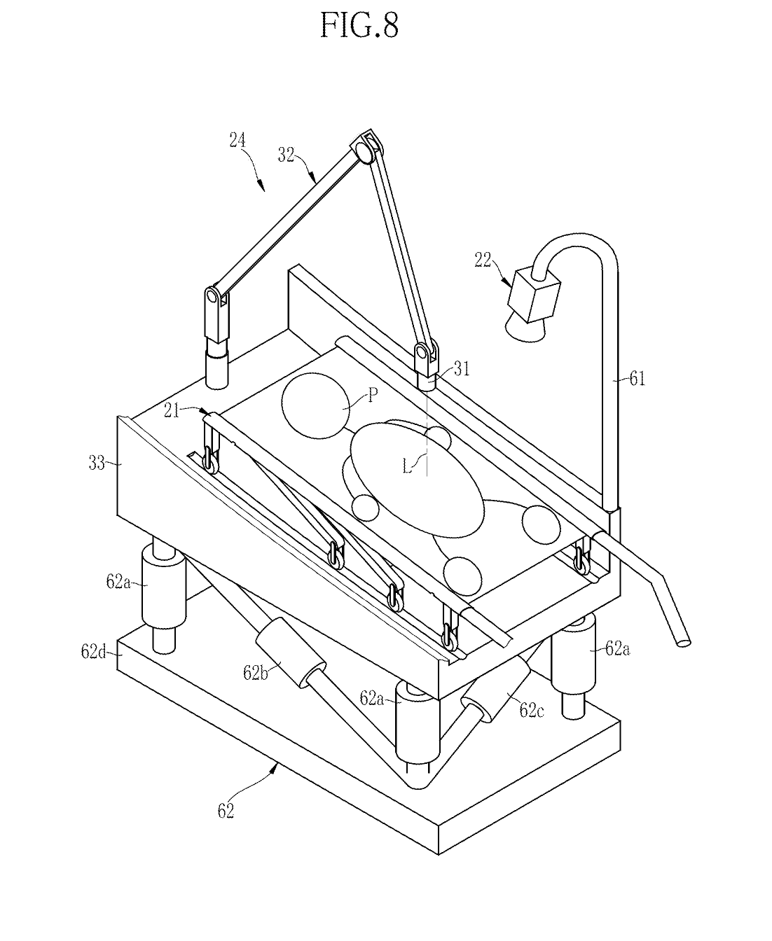

A second embodiment which is another example of the remote indication support system of the present invention will be described below in detail. The second embodiment is different from the first embodiment in that a photographing unit fixation unit 61 and a damping device 62 are newly provided as shown in FIG. 8. FIG. 8 is a view in which only a part of the in-vehicle device 12 of the second embodiment is shown, and the control device or the remote indication device 14 is omitted. The photographing unit fixation unit 61 fixes the photographing unit 22 to the bed fixing base 33. The damping device 62 is provided on the lower side of the bed fixing base 33. The same configurations and functions as the above-described first embodiment will be given the same reference numerals, and the detailed description thereof will not be repeated.

The damping device 62 is constituted of substantially rod-like 8 oil dampers 62a, 62b, and 62c; and a substantially rectangular plate 62d which is provided so as to be opposed to the surface on the lower side of the bed fixing base 33. The four oil dampers 62a are substantially vertically fixed to the surface on the lower side of the bed fixing base 33. All of the other ends of the four oil dampers 62a are fixed to the plate 62d. The two oil dampers 62b are provided on the surface on the lower side of the bed fixing base 33 diagonally in a longitudinal direction. The two oil dampers 62b are arranged at a position twisted from each other. The Two oil dampers 62c are provided on the surface on the lower side of the bed fixing base 33 diagonally in a lateral direction. The two oil dampers 62c are arranged at a position twisted from each other.

Well known oil dampers are used as all of the oil dampers 62a, 62b, and 62c. Here, all of the oil dampers 62a, 62b, and 62c have a structure in which a spring seat supporting a spring is provided on a shock absorber. Here, the shock absorber is an expandable cylinder damper, and is an oil type (liquid type) using fluid resistance of an incompressible liquid. The shock absorber generates resistance and obtains damping force through movement of a fluid due to a piston which moves in accordance with the expansion and contraction of the shock absorber. The spring absorbs an impact by being elastically deformed. Accordingly, all of the oil dampers 62a, 62b, and 62c can absorb impacts with respect to directions, in which the oil dampers are respectively provided, and damp vibrations generated by the impacts.

The oil dampers 62a can absorb impacts in a substantially vertical direction on the surface on the lower side of the bed fixing base 33. In addition, all of the oil dampers 62b and 62c are provided in directions which are not parallel to the oil dampers 62a. Therefore, in the oil dampers 62a, it is possible to absorb impacts in directions which could not be absorbed. For this reason, the damping device 62 can reliably absorb impacts which are generated in the ambulance 11 and are transmitted to the bed fixing base 33.

In the first and second embodiments, the light pointer 24 and the stretcher 21 are fixed to the bed fixing base 33, and therefore, the relative position between the light pointer 24 and the stretcher 21 does not change due to a vibration of the ambulance 11, which is preferable. Furthermore, in the second embodiment, the photographing unit 22 is also fixed to the bed fixing base 33, and therefore, neither the relative position between the photographing unit 22 and the stretcher 21 nor the relative position between the photographing unit 22 and the light pointer 24 change due to a vibration of the ambulance 11, which is more preferable.

In the second embodiment, the damping device 62 has been made to include oil dampers 62a, 62b, and 62c. However, the present invention is not limited thereto, and any mode may be used as long as impacts from the ambulance 11 are absorbed. For example, magnetic dampers (refer to JP2012-205872) can be used instead of the oil dampers.

### Third Embodiment

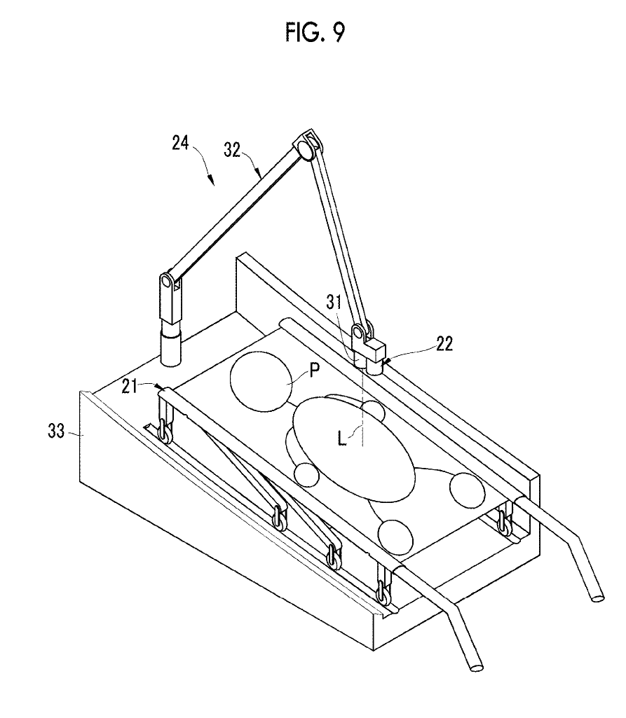

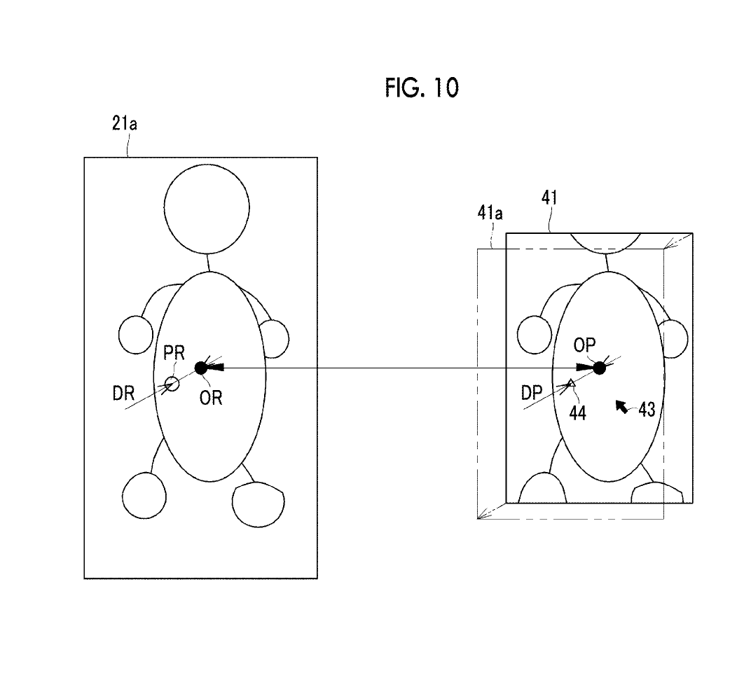

A third embodiment which is another example of the remote indication support system of the present invention will be described below in detail. The third embodiment and the first embodiment are different from each other in that the irradiation unit 31 and the photographing unit 22 are adjacently attached to a distal end of the arm 32d as shown in FIG. 9. Since the irradiation unit 31 and the photographing unit 22 are attached to the distal end of the arm 32d, the irradiation unit 31 and the photographing unit 22 move together through the movement of the arm 32d. In addition, the irradiation unit 31 and the photographing unit 22 are at almost the same position. Therefore, as shown in FIG. 10, the central position OP of a photographed image photographed by the photographing unit 22 and the irradiation position of the irradiation unit 31 substantially correspond to each other at all times.

Similarly to the first embodiment, in the remote indication device 14, in a case where the position shown by the mark 44 of the photographed image 41 is designated as a movement destination of the irradiation unit 31 through a position designation operation, the coordinate transformation unit 52 transforms the designation of this movement destination from the coordinate within an image into the actual coordinate. The first control unit 51 moves the irradiation unit 31 to the position PR by operating the displacement mechanism 32 based on a movement indication after the transformation in which the movement destination is designated by the actual coordinate. At this time, in the third embodiment, the photographing unit 22 is also moved to the vicinity of the position PR at the same time as the movement of the irradiation unit 31. Accordingly, the central position OP of the photographed image is also moved to the position shown by the mark 44. The portion of the region which is surrounded by an imaginary line is newly displayed on the display 37 as the photographed image 41a.

In the third embodiment, the irradiation position substantially comes to the center of the photographed image, and therefore, there is an advantage that the doctor D easily intuitively checks the indicated position with respect to the rescue worker C. With such a configuration, the displacement mechanism 32 does not interfere the photographing of the photographing unit 22, for example, the arm 32d being photographed within the photographed image 41. For this reason, the photographing unit 22 can photograph the photographed image of the patient P without any blind spot caused by the displacement mechanism 32. Accordingly, the doctor D easily indicates a portion which becomes a blind spot due to the displacement mechanism 32, which is preferable.

The present invention can also be used in an inspection other than the ultrasonic inspection, for example, in an inspection through X-ray photography in which a cassette type digital X-ray photographic device is used. However, in the present invention, it is possible to designate a fine position through irradiation with laser light. Therefore, a case of performing an inspection using an ultrasonic wave within a comparatively narrow range is more effective than a case of performing an inspection through X-ray photography within a comparatively wide range. In addition, an inspection site using an ultrasonic wave is fine, and therefore, the present invention is particularly effective in the case of FAST which is performed in emergencies.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. A remote indication support system for supporting a remote indication which is related to an operation of a medical device and is performed from a remote place for an operator who operates the medical device, the remote indication support system comprising:
A. an in-vehicle device in which the medical device is provided and which is provided within a transport vehicle for transporting a patient, the in-vehicle device including:
a photographing unit for photographing the patient;
a photographed image transmission unit for transmitting a photographed image which has been photographed by the photographing unit to the remote indication device;
an indication reception unit for receiving an indication relating to an inspection site, in which an inspection is performed by the medical device, from the remote indication device; and
a light pointer for irradiating the patient with light so as to point to the inspection site, the light pointer being adapted to displace the position of emitting the light based on the indication which has been received by the indication reception unit; and
B. a remote indication device which is connected to the in-vehicle device through an communication network so as to be communicable with each other, the remote indication device including:
a photographed image display unit for displaying the photographed image which has been received from the photographed image transmission unit;
a position designation reception unit for receiving an input of a position designation operation for designating a position of the inspection site from a body of the patient within the photographed image;
an indication generation unit for generating the indication based on the position designation operation received by the position designation reception unit; and
an indication transmission unit for transmitting the indication generated in the indication generation unit to the in-vehicle device.

2. The remote indication support system according to claim 1, further comprising a coordinate transformation unit for transforming information of a coordinate within an image in the photographed image designated through the position designation operation, into information of an actual coordinate for controlling the position of emitting the light using the light pointer.

3. The remote indication support system according to claim 1, wherein the light pointer has an irradiation unit for irradiating laser light, and a displacement mechanism for displacing the irradiation unit.

4. The remote indication support system according to claim 1, wherein the photographing unit is an optical camera which photographs the patient.

5. The remote indication support system according to claim 1,
wherein the medical device is an ultrasound diagnostic apparatus which has a probe to be brought into contact with the body of the patient, and generates and displays an ultrasound image based on a signal from the probe, and
wherein the inspection site is a site to be brought into contact with the probe.

6. The remote indication support system according to claim 5, which is used at the time of performing FAST, as prompt and easy ultrasonography, using the ultrasound diagnostic apparatus.

7. The remote indication support system according to claim 1,
wherein the remote indication device has a current position reception unit for receiving a current position of the position of emitting the light using the light pointer, from the in-vehicle device, and
wherein the current position is superimposed on the photographed image and displayed in the photographed image display unit.

8. The remote indication support system according to claim 1, wherein the in-vehicle device has a damping device which is provided to a bed fixing base for fixing a bed, on which the patient is laid down, and removes a vibration transmitted to the bed from the transport vehicle.

9. The remote indication support system according to claim 8, wherein at least one of the photographing unit and the light pointer is fixed to the bed fixing base.
